(19) United States
(12) Patent Application Publication
(10) Pub. No.: US 2022/0347333 A1
(43) Pub. Date: Nov. 3, 2022

(54) PORTABLE DRY SANITIZING COMPARTMENT

(71) Applicant: Amy Monaco, Oro Valley, AZ (US)

(72) Inventor: Amy Monaco, Oro Valley, AZ (US)

(21) Appl. No.: 17/735,189

(22) Filed: May 3, 2022

Related U.S. Application Data
(60) Provisional application No. 63/183,418, filed on May 3, 2021.

(51) Int. Cl. A61L 2/26; A61L 2/10; A61L 2/22; D06F 58/10; D06F 34/10; D06F 58/20

(52) U.S. Cl. CPC A61L 2/26; A61L 2/10; A61L 2/22; D06F 58/10; D06F 34/10; D06F 58/203; A61L 2202/16; A61L 2202/122; A61L 2202/14; A61L 2202/11; A61L 2202/15; A61L 2202/26

(57) ABSTRACT

A portable modular sanitizing compartment is provided that is designed and configured to facilitate rapid sanitizing and drying of clothing articles placed within the compartment. The sanitizing compartment may include, an exterior housing, modular base controller and internal compartment defining an interior space, a plurality of air intake valves, a plurality tubular structure with plurality of apertures connected to hangers within the internal compartment, a plurality of modular UV lights, and a modular base control unit to form a treatment compartment. The use of treated atmospheric air, fragrance or atomized disinfectant instead of ozone to treat the clothing from the inside out through a structure of connected tubes placed within a closed compartment may provide the user with a better use case for users by limiting adverse health effects. The portable dry sanitizing compartment may also be controlled externally through a smart device.

### FIELD OF THE INVENTION

The present invention relates generally to an apparatus and method for sanitizing and drying clothing. More particularly, the present invention relates to a modular portable dry sanitizing compartment that may utilize a combination of techniques such as purified air and/or ozone along with either ultraviolet light or light emitting diodes to both dry and sanitize the contents within the compartment with removable elements.

### BACKGROUND

The use of ozone (O3) is well known as a disinfectant and a sanitizing agent. Chemically, ozone is considered an unstable gas because of its ability to readily degrade back to diatomic oxygen (O2) also known as atmospheric oxygen. During the degradation process, free oxygen atoms, also known as free radicals, are formed and those free radicals are capable of oxidizing a variety of substances including: viruses, fungi, mold, and bacteria. As a result, the oxidation properties of ozone are not only able to serve as a disinfectant, but may also serve as a sanitizer that is capable of eliminating odors.

The oxidation properties, as well as the penetrative properties, of ozone make it and ideal aerial and surface disinfectant. Ozone is commonly used in cleaning a variety of equipment such as furniture, carpets, and garments. While ozone may be an ideal candidate for the reasons described above, the use of ozone as a sanitizing agent does not come without drawbacks. Firstly, because ozone is a very unstable and highly reactive form of oxygen it may pose some adverse effects on human health. Acute ozone exposure may occur with persistent use of ozone as a sanitizing agent. Inhaled ozone causes inflammation and acute—but reversible-changes in lung function, as well as airway hyperresponsiveness. These changes may lead to shortness of breath, wheezing, and coughing which may exacerbate lung diseases, like asthma or chronic obstructive pulmonary disease (COPD) resulting in the need to receive medical treatment. Consequently, alternatives to ozone may be desired when considering sanitizing agents.

Similar to ozone, the use of ultraviolent (UV) light has also been commonly used as a disinfectant and sanitizing agent. UV light is a form of light that is invisible to the human eye. It occupies the portion of the electromagnetic spectrum between X-rays and visible light. A unique characteristic of UV light is that a specific range of its wavelengths, those between 200 and 300 nanometers (billionths of a meter), are categorized as germicidal—meaning they are capable of inactivating microorganisms, such as bacteria, viruses and protozoa. As a result, UV light has been used for sterilization and disinfection as early as the mid-20th century. With advancements in technology, and specifically in the UV bulbs themselves, its reliable long lifespan (thousands-of-hours) and smaller size (e.g. UV LED vs traditional UV bulbs) has broadened the field for where it can be used. This capability has allowed widespread adoption of UV light as an environmentally friendly, chemical-free, and highly effective way to disinfect and sanitizing a variety of things such as water, air, produce, surgical utensils, and surfaces.

Sports equipment, as well as hunting equipment, has a reputation for becoming saturated from sweat, drying and eventually producing unpleasant odors. The unpleasant smelling sports equipment, and the space necessary to dry it, is currently an ongoing source of conflict in many household or traveling situations in confining spaces or remote areas. Also, in the instance of athletics many games are played soon after each other, a player often does not have enough time to properly dry the equipment before the next use. This makes for a clammy and unpleasant feeling; putting on wet, damp, or dirty equipment. In addition, if not properly dried or cleaned, the sweat-soaked equipment becomes a site for growth of bacteria, mold, mildew, fungus, and other microorganisms that can spread disease, cause odor and/or damage or discolor the equipment.

Usually, damp garments, sport and athletic equipment, boots and shoes are removed from a sports bag, and spread out to dry out in locker rooms, hotels rooms or storage areas having inadequate air circulation. When the equipment is seemingly dry, the equipment is placed back into the sports bag which creates a closed environment. For unpleasant odors, it is common practice to place aroma packs, air fresheners and the like into the sports equipment bag. However, if the sports equipment is not removed from the bag to dry, the effect of the air fresheners is greatly minimized. Additionally there is no active air flow to hep permeate the active chemical agents in the air fresheners through the clothing or gear.

Along with sports equipment, another common issue within the realm of clothing and garment sanitization is the predicament of cleaning clothes while travelling. Often times when people are travelling for prolonged periods of time, they may not have access to a means for washing their clothes. In such situations, it may also be common for people to wear the same clothes for multiple days because they may only have access to a limited portion of their wardrobe while travelling due to travelling and convenience constraints.

Many solutions to the aforementioned problems tend to create other issues. For instance, sanitization bags that utilize ozone that comes into direct contact with clothing may destroy or seriously damage the materials and/or fabrics from which the equipment is made. Additionally, persistent exposure to ozone as it is released from the bag may cause adverse health effects. Other solutions may require complex cleaning units that may be quite large and bulky, not making them suitable to be used portably.

Accordingly, there remains a need in the art for a solution to at least one of the aforementioned problems. For instance, a modular sanitization compartment that is able to quickly and efficiently sanitize and dry clothing while limiting and/or omitting the use of ozone is desired. Additionally, it is desired that the compartment be include modular features to customize the methods for treating garments and is suitable to be used portably.

### SUMMARY OF THE INVENTION

The present invention is directed to a portable dry sanitizing compartment that facilitates rapid sanitizing and drying of any garment placed inside a modular compartment. The portable dry sanitizing modular compartment includes an exterior surface, a modular base unit and structure along with an interior compartment. The portable dry sanitizing compartment may further include a plurality of air intake valves pulling air from outside the compartment into the interior compartment of the compartment. Additionally, the interior of the modular compartment may also include a plurality of tubing and hangers for hanging clothes that push deodorizing agents from the inside out of clothing to be sanitized and dried using the atmospheric air channeled inside the compartment. This method is superior to external treatment in of clothing as the sweat occurs on the inside that comes in contact with the skin not necessarily on the outside of the garments. The compartment may also include a plurality of modular UV LED lights in the interior compartment of the compartment such that may be customize to further sanitize the garments placed inside the compartment of different types and sizes. Additionally a modular feature to accept the use of a the gas cylinder hoisting apparatus is also provided.

The portable modular sanitizing compartment may solve a number of problems currently associated with rapid cleaning and drying solutions for garments. Firstly, the portable modular sanitizing compartment does not require any complex, large, or bulky exterior equipment. As a result, the compartment is readily designed for portable use and may be used in a variety of contexts, ranging from department store applications, to a traveling accompaniment, to a rapid cleaning solution for sports teams. Secondly, the use of atmospheric air instead of chamber can infuse ozone or atomized disinfectant pushing the treatments from the inside out of the clothing via a modular tubular structure providing the user with a better use case for the portable modular sanitizing compartment in terms of limit adverse health effects. Lastly, the portable modular sanitizing compartment may be controlled externally from a smartphone device or other application through Bluetooth technology. This allows a user to control sanitization and drying cycles externally without needing to open the compartment and optimize the treatments based on the material type and sizes making for a smart sanitization system.

In a first implementation of the invention, a portable modular sanitizing compartment configured to sanitize and dry garments placed within the compartment, the portable dry sanitizing compartment comprises:

an exterior modular housing, the exterior housing forming an outer surface of the compartment;
an internal compartment within the exterior housing, the internal compartment defining an interior space configured to receive one or more articles of clothing; wherein the internal compartment further includes: a plurality of air intake valves configured to pull atmospheric air from outside the compartment; a tubular structure with integrated hangers each connected to each of the plurality of fan or disinfecting atomizer with valves to direct treated air through clothing, the tubular structure and plurality of hangers is perforated uniformly allowing ozone or atomized disinfectant to permeate clothing from the inside out plurality of hangers configured to hang the one or more articles of clothing, the plurality of hangers further including a plurality of apertures configured to disperse the atmospheric air or disinfectant from the plurality of air intakes or disinfecting atomizer through one or more articles of clothing;
a plurality of modular UV lights configured to sanitize the one or more articles of clothing; and
a control unit having a battery and a PCB board, wherein the control unit is configured to turn on and off the plurality of intake valves and the plurality of UV lights.

In a second aspect, the exterior housing may include an openable panel, wherein the openable panel provides access to the internal compartment and the interior space. This housing is soft material that creates a barrier of trapping treated air or atomized disinfectant withing the chamber. Next the soft material is ideal for collapsing into a compact form allowing for portability.

In another aspect, the openable panel may include a top surface, wherein the top surface defines a top wall of the exterior housing.

In another aspect, the openable panel may include a bottom surface, wherein the bottom surface connects to the modular power unit that stores the fan, motor or disinfecting atomizer chamber and removable tank.

In another aspect, the exterior housing may include a proximal end and a distal end, wherein the proximal end may be semi-circle shaped and the distal end may be rectangular shaped.

In another aspect, the exterior housing may have a plurality of walls forming the top wall and a bottom wall connected by a plurality of sidewalls.

In another aspect, the plurality of side walls may include a plurality of vents.

In another aspect, the plurality of vents may be disposed about the proximal end of the exterior housing.

In another aspect, the plurality of side walls may include a plurality of air intake holes, wherein the air intake holes are disposed about the distal end of the exterior housing, and further wherein the air intake holes lead to the air intake valves within the interior space of the internal compartment.

In another aspect, the internal compartment may include a base surface, wherein the base surface forms an opposite of the bottom wall of the exterior housing.

In another aspect, the base surface may be rigid.

In another aspect, the plurality of air intake valves may further include an inlet fan and a heating coil.

In another aspect, the inlet fan may be configured to suction atmospheric air from outside the compartment through the air intake valves.

In another aspect, the heating coil may be configured to heat the atmospheric air taken in by the air intake valves.

In another aspect, the internal compartment connects to a modular based that may include a removable disinfecting tank and atomizing element.

In another aspect the modular base would include a fragrance tray, wherein the fragrance tray is configured to mix fragrance with the heated atmospheric air.

In another aspect, an internal structure of modular tubing with perforated apertures direct treated air through clothing inside out and allow for disassembly for portability.

In another aspect, the internal tubular structure is connected to a plurality of hangers,
In another aspect, the plurality of hangers may include a first portion and a second portion.
In another aspect, the first portion may include an inverted U-member and a vertical member connected to the center of the inverted U-member.

In another aspect, the first portion may be configured to allow a shirt to be hung.

In another aspect, the second portion may include a first member vertical member and a second vertical connected by a horizontal member.

In another aspect, the horizontal member may include a pair of L-shaped clips extended outward from the horizontal member.

In another aspect, the second member may be configured to allow a pair of pants to be hung.

In another aspect, the plurality of hangers may be comprised of hollow tubing.

In another aspect, the pair of L-shape clips may be configured to clip to a waist portion of the pair of pants.

In another aspect, the internal compartment may include a plurality of sidewalls, wherein the plurality of sidewalls are opposite the plurality of sidewalls of the exterior housing.

In another aspect, the plurality of UV lights may be disposed about the plurality of side walls of the internal compartment.

In another aspect, the plurality of modular UV lights may be LED UV lights.

In another aspect, the control unit may include Bluetooth connectivity.

In another aspect, the control unit may be controlled through an external smart device through the Bluetooth connectivity.

In another implementation of the invention, a method of operating the portable dry sanitizing compartment comprises obtaining a portable dry sanitizing compartment, the portable dry sanitizing compartment having an exterior housing, the exterior housing forming an outer surface of the compartment, an internal compartment within the exterior housing, the internal compartment defining an interior space configured to receive one or more articles of clothing, wherein the internal compartment includes a plurality of air intake valves configured to pull atmospheric air from outside the compartment; a plurality of hangers each connected to each of the plurality of air intake valves, the plurality of hangers configured to hang the one or more articles of clothing, the plurality of hangers further including a plurality of apertures configured to disperse the atmospheric air from the plurality of air intake valves on to the one or more articles of clothing, a plurality of modular UV lights configured to sanitize the one or more articles of clothing; and a control unit having a battery and a PCB board, wherein the control unit is configured to turn on and off the plurality of intake valves, disinfecting tank, atomizer and the plurality of UV light; turning on the control unit through at least one of the control unit itself and a smart device; inserting a fragrance tray into the internal compartment; setting a heating temperature; setting a cycle time inserting one or more articles of clothing into the internal compartment, wherein the one or more articles of clothing are hung about the hangers; and starting the control unit, wherein starting the control unit turns on the UV lights and further wherein turning on the control unit allows air intake suction through the air intake valves and the air is heated, mixed with fragrances, and dispersed through the plurality of hangers.

In another aspect, the one or more articles of clothing may include both a shirt and a pair of pants.

In another aspect, the heating temperature may be set between 125°-150° F.

In another aspect, the cycle time may range from 15 minutes to 60 minutes.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a front perspective view of a portable dry sanitizing compartment, shown with the openable panel in an open state, in accordance with a first illustrative embodiment of the invention;

FIG. 2 presents a front perspective view of the portable dry sanitizing compartment illustrated in FIG. 1, shown with emphasis to the vents, air intake valves, and UV lights;

FIG. 3 presents a front perspective view of the plurality of hangers of the portable dry sanitizing compartment illustrated in FIG. 1;

FIG. 4 a presents a top plan view of the portable dry sanitizing compartment illustrated in FIG. 1, shown in an open configuration wherein the control unit of the compartment may be controlled by a smart device through Bluetooth connectivity;

FIG. 5 presents a schematic of the components of the portable dry sanitizing compartment illustrated in FIG. 1; and

FIG. 6 presents a flowchart of a method of use of the portable dry sanitizing compartment illustrated in FIG. 1. and

FIG. 7 presents an alternative schematic of the components of the portable dry sanitizing compartment illustrated with a disinfecting tank, and atomize in FIG. 1.

Like reference numerals refer to like parts throughout the several views of the drawings.

### DETAILED DESCRIPTION OF AN EXEMPLARY EMBODIMENT

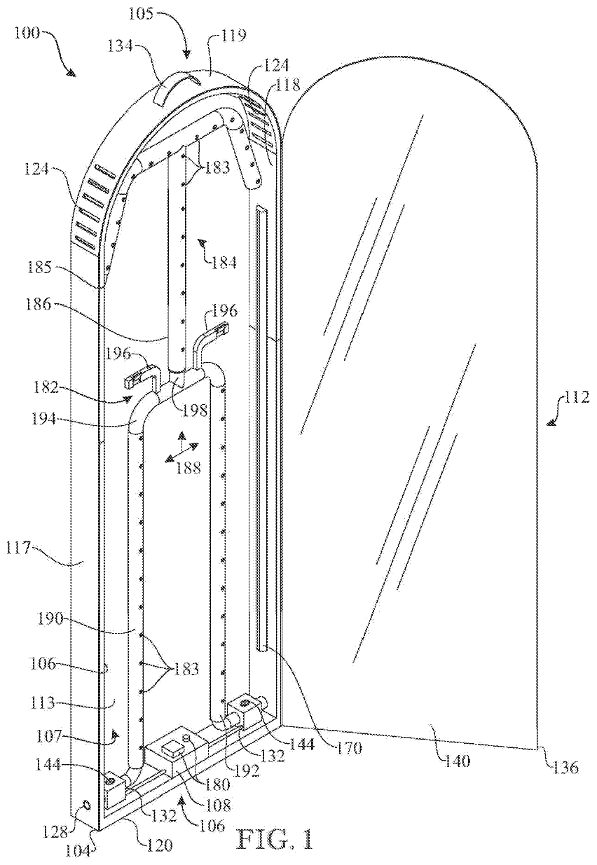

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward portable dry sanitizing compartment that is designed and configured to facilitate rapid sanitizing and drying of clothing articles placed within the compartment. The sanitizing compartment may include, an exterior housing, and internal compartment defining an interior space, a plurality of air intake valves, an internal tubular structure to direct treated air through clothing, a plurality of hanger within the internal compartment, a plurality of modular UV lights, and a control unit for the compartment. The portable dry sanitizing compartment operates by intaking atmospheric air from the exterior of the compartment, heating and mixing that air with a fragrance, and dispersing that air over any article of clothing hung within the compartment. Additionally, the placement of the plurality of UV lights within the compartment serve to kill bacteria an sanitize the articles of clothing placed within the compartment. (I realize the drawings don't show clothes around the tubing which is important to communicate the innovation—how should we handle this? I can modify the drawings)

Referring initially to FIG. 1, a portable dry sanitizing compartment 100 is illustrated in accordance with an exemplary embodiment of the present invention. As shown, the sanitizing compartment 100 has a and exterior housing 104 forming an outer surface of the sanitizing compartment 100 and an internal compartment 106 defining an interior space 107 of the sanitizing compartment 100. The sanitizing compartment 100 further includes a plurality of components within the internal compartment 106 of the compartment 100, as will be described in greater detail hereinafter. The sanitizing compartment 100 may further include a control unit 108 for operating each of the internal components.

With continued reference to FIG. 1, the exterior housing 104 may include a proximal end 105 and a distal end 106. The proximal end 105 may be a semi-circle shaped and the distal end 106 may be rectangular shaped. The exterior housing 104 may include a plurality of walls forming a top wall 112 and a bottom wall 113, connected by a plurality of sidewalls 117, 118, 119, 120. The plurality of sidewalls 117, 118, 119, 120 may include a first sidewall 117 and a second sidewall 118, which extend the length of the exterior housing. Additionally, the plurality of sidewalls 117, 118, 119, 120 may further include a third sidewall 119 which may be semi-circle shaped and may sit at the proximal end of the exterior housing 104. A fourth sidewall 120 may sit at the distal end 106 of the exterior housing 104. The plurality of sidewalls 117, 118, 119, 120 may include a plurality of vents 124 at the proximal end 105 of the sanitizing compartment 100. More particularly, each of the plurality of vents 124 may sit on the third sidewall 119 of the exterior housing 104. The plurality of vents 124 may each be configured as a plurality of slits or small, open spaces. The plurality of vents 124 may be positioned such that each of the plurality of vents 124 sits on each side of the third sidewall 119, wherein each of the plurality of vents are symmetrically opposed to each other. Near the distal end 106 of the exterior housing, on the first sidewall 117 and the second sidewall 118, may be a plurality of air intake holes 128, wherein the plurality of air intake holes 128 lead to a plurality of air intake valves 132 in the interior compartment 106 of the sanitizing compartment 100. Also affixed at the center of the third sidewall 119, at the proximal end 105 the exterior housing 104 may be a handle 134. The handle 134 may be configured to allow the sanitizing compartment 100 to be held and carried when the compartment 100 is in a closed configuration.

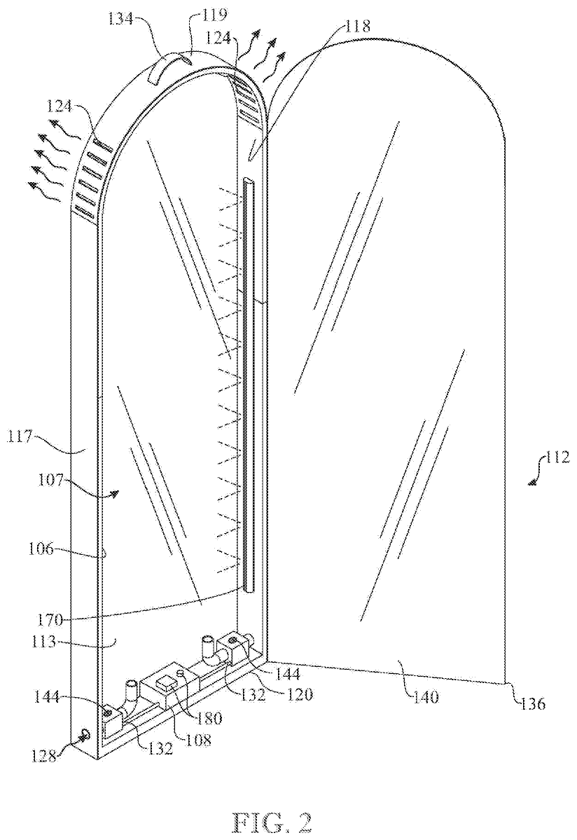

Referring now to FIG. 2, spanning the length of the exterior housing 104, may be an openable panel 136. The openable panel 136 may configured to provide access to the interior compartment 106 and the interior space 107 of the sanitizing compartment 100. The openable panel 136 may include atop surface and a bottom surface 140. The top surface may serve as the top wall 112 of the exterior housing 104. The bottom surface 140 may rest within the internal compartment 106 when the sanitizing compartment 100 is in a closed configuration. The openable panel 136 may have an edge 142 that remains affixed to one of the plurality of sidewalls 117, 118, 119, 120. In the preferred embodiment, the edge 142 of the openable panel 136 may remain affixed to the second sidewall 118, however embodiments are envisioned in which the edge 142 of the openable panel 136 may remain on the first sidewall 117. The openable panel 136 may be flexible and may open through a fastening mechanism such as a zipper. Alternatively, the openable panel 136 may be rigid and may be treated more like a door, in which case the fastening mechanism may be a latch.

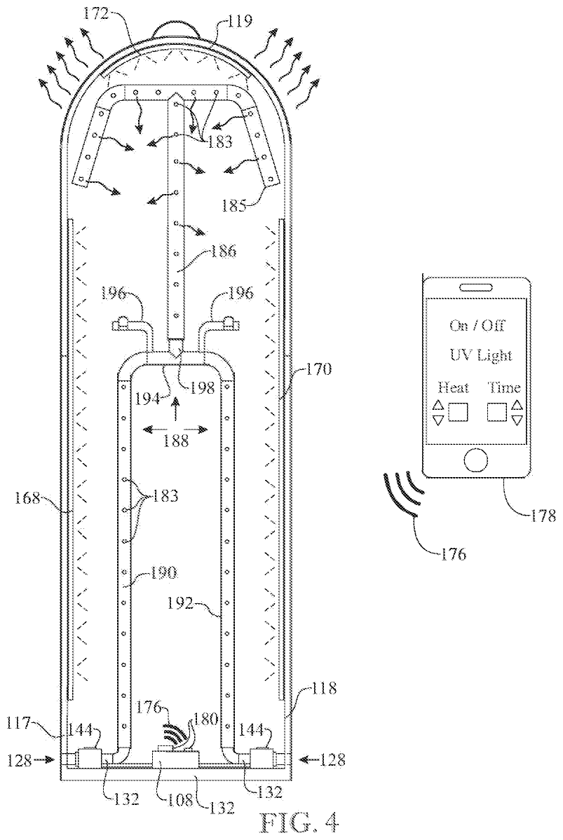

Referring now to FIG. 4, the internal compartment 106 includes the aforementioned plurality of components. Like the exterior housing 104, the internal compartment 106 may include the same proximal end 105 and a distal end 106. Also like the exterior housing 104, the internal compartment 106 may also have the same plurality of walls, wherein the each of the plurality walls has a surface that lies within the interior space 107 of the internal compartment 106. For instance, the bottom wall 113 of the exterior housing 104 may form a base surface of the internal compartment 106, wherein one side of the base surface lies outside the internal compartment 106, and another surface lies within the interior space 107 of the internal compartment 106. The bottom wall 113 is preferably rigid so as to hold the plurality of components within the interior space 107.

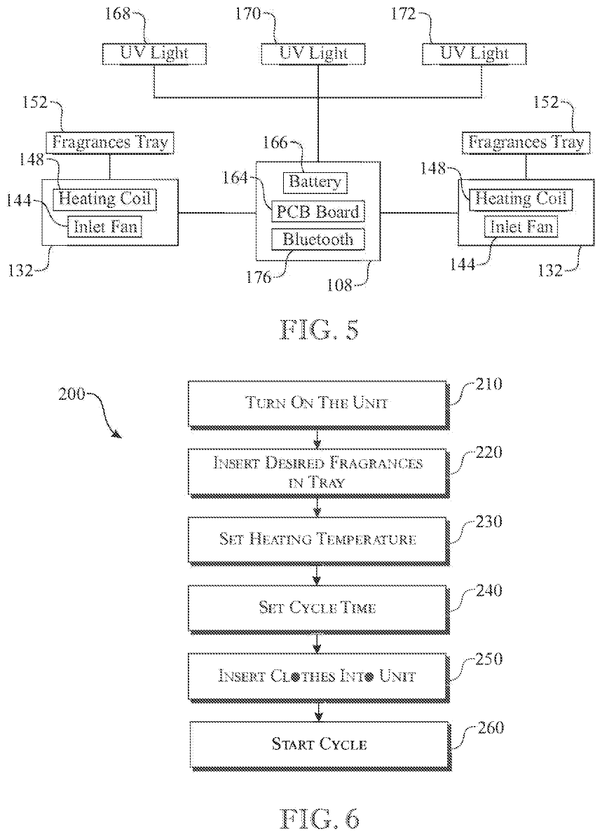

With continued reference to FIG. 4, at the distal end 106 of the internal compartment 106, along an interior surface of the fourth sidewall 120, may be the aforementioned plurality of air intake valves 132. In the preferred embodiment, there may be two air intake valves 128 which lead out to the aforementioned air intake holes 128 on the first sidewall 117 and the second sidewall 118. Each of the plurality of air intake valves 132 may include an inlet fan 144 and a heating coil 148, as best shown in FIG. 5. Each of the plurality of air intake valves 132 may be configured to suction atmospheric air from outside the compartment 100, through the suction capability of the inlet fan 144. The heating coil 148 may be configured to hear the atmospheric air suctioned in from the inlet fan 144. Additionally, each of the plurality of air intake valves 132 may further include a fragrance tray 152. The fragrance tray 152 may be configured to mix a desired fragrance with the heated air that has been suction in and heated by inlet fan 144 and the heating coil 148, respectively.

Referring back to FIG. 4, each of the plurality of air intake valves 132 may be electrically coupled with the aforementioned control unit 108. The control unit 108 may include a battery 160 and a PCB board 164, as shown in FIG. 5. The control unit 108 may be configured to turn on and off the plurality of air intake valves 132, as well a plurality of UV lights 168, 170, 172 dispersed throughout the internal compartment 106 of sanitizing compartment 100, which will be described in greater detail hereinafter. Additionally, the control unit 108 may include Bluetooth connectivity 176, wherein the Bluetooth connectivity 176 is used to operate the sanitizing compartment 100 through a smart device 178, as shown in FIG. 4. Alternatively, the control unit 108 may also have buttons 180 for controlling the compartment 100 internally.

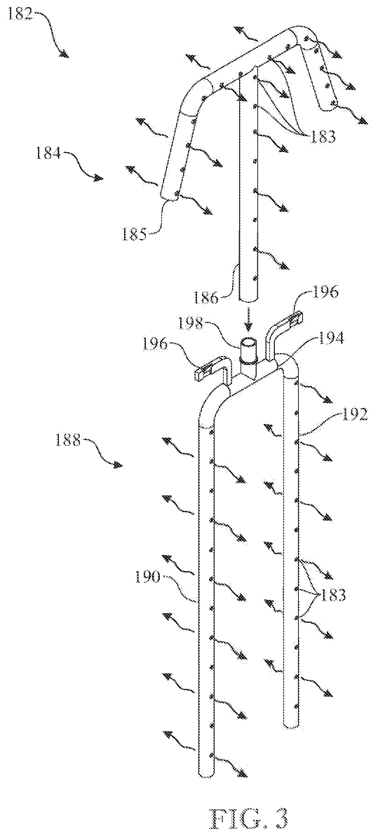

With continued reference to FIG. 4, the plurality of air intake valves 132 may be connected to a plurality of hangers 182. The plurality of hangers 182 may be configured to hang one or more articles of clothing Referring now to FIG. 3, the plurality of hangers 182 may be comprised of hollow tubing. The hollow tubing may be rigid. Alternatively, the hollow tubing may be somewhat malleable. Embodiments are envisioned wherein the hollow tubing of the plurality of hangers 182 may be comprised of metal or metal alloy, or some type of plastic. Additionally, the plurality of hangers 182 may include a plurality of apertures 183 configured disperse the heated and fragranced atmospheric air on to the hung one or more articles of clothing. The plurality of hangers 182 may include a first portion 184 and a second portion 188. The first portion 184 may include an inverted U-member 185 and a vertical member 186 connected to a center of the inverted U-member 185. The first portion 184 may thus resemble a T-shape and may be configured to allow a shirt to be hung thereon.

With continued reference to FIG. 3, the second portion 188 of the plurality of hangers 182 may a first vertical member 190 and a second vertical member 192, wherein the first vertical member 190 and the second vertical member 192 are connected by a horizontal member 194. The horizontal member 194 may further include a pair of L-shaped clips 196 and a circular attachment piece 198. The attachment piece 198 may be configured to connect the second portion 188 to the first portion 184 of the plurality of hangers 182. The second portion 188 of the plurality of hangers 182 may be configured to allow a pair of pants to be hung. Additionally, the L-shaped clips may be configured to clip on to a waist portion of the pair of pants.

Referring back to FIG. 4, the aforementioned plurality of UV lights 168, 170, 172 may be affixed to the plurality of sidewalls 117, 118, 119, 120. More particularly, a first UV light 168 may be affixed to an interior portion of the first sidewall 117. A second UV light 170 may be affixed to an interior portion of the second sidewall 118. A third UV light 172 may be affixed to the third sidewall 119. The third UV light 172 may be curved to conform to the shape of the third sidewall 119. Each of the plurality of UV lights 168, 170, 172 may be UV LED lights. The plurality of UV lights 168, 170, 172 may be configured to sanitize the one or more articles of clothing hung about the plurality of hangers 182. As mentioned previously, the UV LED lights may also be controlled by the control unit 108.

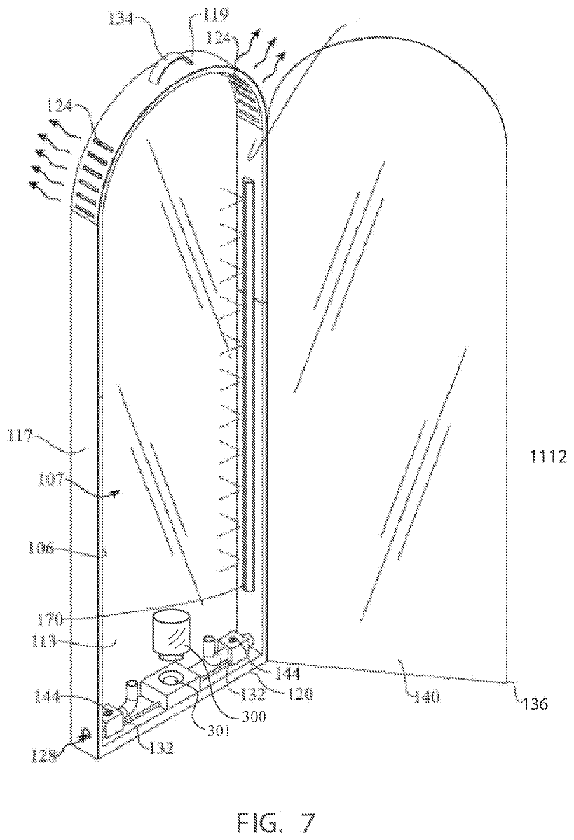

Additionally in FIG. 7 the method of sanitizing the clothing can take place with a atomizing disinfectant liquid using a atomizer. The disinfectant is stored in a removable tank 300 and is inserted into a atomizing receptacle 301 at the bottom of the sanitizing compartment. Within this receptable is an electrical element that when energized atomizes the liquid that is dispensed into the atomizing chamber.

The flowchart 200 of FIG. 6 may demonstrate an example method of operation for the portable dry sanitizing compartment 100. The example method is described below with reference to the flowchart depicted in FIG. 6, as well as the illustrations of FIGS. 1-5, as a series of steps (steps 210, 220, 230, 240, 250, 260) that may be altered or varied depending on a variety of factors, such as the types of clothing articles to be sanitized or the type of sanitization that is desired.

The method may begin by step 210, wherein the sanitizing compartment 100 is turned on. This may be done directly through the buttons 180 on the control unit 108, as shown in FIG. 4. Alternatively, a user may turn on the compartment 100 utilizing their smart device 178 through Bluetooth connectivity 176. Turning on the compartment 100 may entail both starting the plurality of air intakes vales 132, such that the air intake valves 132 begin suctioning air through the inlet fan 144, as well as the plurality of UV lights 168, 170, 172. Both the plurality of air intake valves 132 and the plurality of UV lights 168, 170, 172 may only start when the compartment is in a closed configuration. Put another way, the compartment 100 may only start once the openable panel 136 is closed, as shown in FIG. 1.

The next step 220 may involve placing the desired fragrance into the fragrance tray 152. The fragrance may be mixed with the atmospheric air that is being suctioned into the internal compartment 106 of the compartment 100 through the plurality of air intake valves 132. In step 230, a user may set the heating temperature of the compartment 100. The heating temperature may control the heating coil 148 of the air intake valves 132. This may also be controlled through the user's smart device 178. They heating temperature may be, for instance, set to a range from 125°-150° F. However, embodiments are envisioned in which temperatures outside that range may be achieved. In step 240, a user may set the cycle time by which they would like the compartment 100 to run. This may also be controlled through the user's smart device 178. The cycle time may, for instance, range from 15 minutes to 60 minutes. However, embodiments are envisioned in which cycle times outside that range may be achieved.

In step 2500, one or more articles of clothing may be placed into the internal compartment 106 of the compartment 100. If the compartment 100 is in a closed configuration, then access to the interior compartment 106 may be achieved by opening the openable panel 136. Once the openable panel 136 is open, as user may hang the desired clothes on the plurality of hangers 182. More particularly, a user may utilize the first portion 184 to hang a shirt or a top. And the user may utilize the second portion 188 to hang a paint of pants, wherein the L-shaped clips 196 may be used to clip on the waist of a pant. After the clothes are hung within the internal compartment 106 of the compartment 100, in step 260, the user may start the compartment by closing the openable panel 136. Once the compartment 100 has started, air from outside the compartment 100 may be suctioned into the compartment 100, heated, mixed with fragrance, and dispersed over the clothes. Additionally, the plurality of UV lights 168, 170, 172 may be actuated to sanitize the clothes as they are being dried.

Alternative embodiments are contemplated to those shown or described herein without departing from the scope of the present disclosure. For example, embodiments are contemplated in which the compartment may be entirely rigid such that, while portable, may be designed for repetitive fixed use, as in a department store. Another alternative embodiment considered is potentially utilizing various types of lights other than UV lights such as LEDs or other environmentally friendly lights. Lastly, embodiments are envisioned in which the size of the compartment is greatly reduced such that it may be used as an attachment to other compartments or travel items.

In summary, the portable dry sanitizing compartment disclosed herein provides a device which utilizing atmospheric air and UV lights to combine to sanitize clothing placed within the device. The compartment may be small, light, and durable and may be structured to allow a variety of types of clothing to be placed within the compartment, making the compartment portable. Additionally, the compartment and method described herein allow for optimal efficiency by allowing a user to control the compartment through a smart device.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

I claim:

1. A portable modular sanitizing compartment configured to sanitize and dry garments placed within the compartment, the portable dry sanitizing compartment comprising:
an exterior housing, the exterior housing forming an outer surface of the compartment; a modular powered based for the electrical components including fan, electronics and controls:
an internal compartment within the exterior housing, the internal compartment defining an interior space configured to receive one or more articles of clothing; wherein the internal compartment further includes: a plurality of air intake valves configured to pull atmospheric air from outside the compartment; a plurality of hangers each connected to each of the plurality of air intake valves, a modular tubular structure with plurality of apertures to direct treated air through the clothing; the tubular structure is connected to a plurality of hangers with configured to hang the one or more articles of clothing, the plurality of hangers further including a plurality of apertures configured to disperse the atmospheric air from the plurality of air intake valves on to the one or more articles of clothing; (108 we can modify the drawing slightly to show a removable tank for disinfectant—thoughts?)
a plurality of modular UV lights configured to sanitize the one or more articles of clothing; and
a powered modular base unit having a battery and a PCB board, wherein the control unit is configured to turn on and off the plurality of intake valves, disinfecting reservoir, fragrance tray and the plurality of UV lights.

2. The portable dry sanitizing compartment of claim 1 wherein the exterior housing includes an openable panel which provides access to the internal compartment and the interior space; the openable panel includes a top surface which defines a top wall of the exterior housing; and the openable panel includes a bottom surface which is part of the internal compartment.

3. The portable dry sanitizing compartment of claim 2 wherein the exterior housing includes a proximal end and a distal end; the proximal end is semi-circle shaped; and the distal end is rectangular shaped.

4. The portable dry sanitizing compartment of claim 3 wherein the exterior housing has a plurality of walls comprising the top wall and a bottom wall connected by a plurality of sidewalls; wherein the side walls include a plurality of vents disposed about the proximal end of the exterior housing; the side walls include a plurality of air intake holes disposed about the distal end of the exterior housing; and the air intake holes communicate with the air intake valves within the interior space of the internal compartment.

5. The portable dry sanitizing compartment of claim 4 wherein the internal compartment includes a rigid base surface, wherein one side of the base surface lies outside the internal compartment and another side lies within the interior space of the internal compartment.

6. The portable dry sanitizing compartment of claim 5 wherein the plurality of air intake valves comprise an inlet fan configured to suck atmospheric air from outside the compartment through the air intake valves; and a heating coil configured to heat the atmospheric air taken in by the air intake valves.

7. The portable dry sanitizing compartment of claim 6 wherein the internal compartment includes a fragrance tray configured to mix fragrance with the heated atmospheric air.

8. The portable dry sanitizing compartment of claim 7 wherein the plurality of hangers includes a first portion and a second portion; the first portion including an inverted U-member and a vertical member connected to the center of the inverted U-member; and the second portion including a first vertical member and a second vertical member connected by a horizontal member.

9. The portable dry sanitizing compartment of claim 8 wherein the horizontal member includes a pair of L-shaped clips extended outwardly therefrom.

10. The portable dry sanitizing compartment of claim 9 wherein the second member is configured to allow a pair of pants to be hung.

11. The portable dry sanitizing compartment of claim 10 wherein the plurality of hangers is comprised of hollow tubing.

12. The portable dry sanitizing compartment of claim 11 wherein the pair of L-shape clips are configured to clip to a waist portion of the pair of pants.

13. The portable dry sanitizing compartment of claim 12 wherein the internal compartment includes a plurality of sidewalls disposed opposite the plurality of sidewalls of the exterior housing.

14. The portable dry sanitizing compartment of claim 13 wherein the plurality of UV lights is disposed about the plurality of side walls of the internal compartment.

15. The portable dry sanitizing compartment of claim 14 wherein the UV lights are LED UV lights.

16. The portable dry sanitizing compartment of claim 15 wherein the control unit includes Bluetooth connectivity and is controllable through an external smart device through the Bluetooth connectivity.

17. A modular tubular structure with a plurality of apertures that directs treated air into the chamber.

18. A modular tubular structure with a plurality of apertures that direct treated air through clothing from the inside out.

19. A modular powered base enclosed in a compartment that includes a fan, motor, fragrance tray, removable disinfecting tank, atomizer element, air intake, connections to UV LEDs either wires or quick disconnect and electronics. Whereas the removable disinfecting tank is enclosed within the sanitizing compartment.

20. A modular powered based that directs treated air through tubular structure to treat clothing inside out into a soft chamber.

21. A method of operating the portable dry sanitizing compartment comprises: obtaining a portable dry sanitizing compartment, the portable dry sanitizing compartment having an exterior housing, the exterior housing forming an outer surface of the compartment, an internal compartment within the exterior housing, the internal compartment defining an interior space configured to receive one or more articles of clothing, wherein the internal compartment includes:
a plurality of air intake valves configured to pull atmospheric air from outside the compartment; a modular tubular structure with a plurality of apertures, a plurality of hangers each connected to each of the plurality of air intake valves, the plurality of hangers configured to hang the one or more articles of clothing, the plurality of hangers further including a plurality of apertures configured to disperse the atmospheric air from the plurality of air intake valves on to the one or more articles of clothing;
a plurality of modular UV lights configured to sanitize the one or more articles of clothing; and
a modular base control unit having a battery and a PCB board, wherein the base control unit is configured to turn on and off the plurality of intake valves, heater and fan, fragrance tray, hold a removeable disinfecting tank, power disinfecting atomizer and the plurality of UV light;
turning on the control unit through at least one of the control unit itself and a smart device;
inserting the removable disinfecting tank into the modular base with an atomizer;
inserting a fragrance tray into the internal compartment; setting a heating temperature; setting a cycle time;
inserting one of more articles of clothing over the modular tubular structure with a plurality of apertures that direct the treated air through the clothing;
inserting one or more articles of clothing into the internal compartment, wherein the one or more articles of clothing are hung about the hangers; and
starting the modular base control unit, wherein starting the modular base control unit turns on the UV lights and further wherein turning on the modular base control unit allows air intake suction through the air intake valves and the air is heated, mixed with fragrances or atomized disinfectant, and dispersed through the series of connected modular tubes and connected hangers with plurality of apertures.

22. The method of claim 21 wherein the one or more articles of clothing include both a shirt and a pair of pants.

23. The method of claim 22 wherein the heating temperature may be set between 125 F-150 F.

24. The method of claim 23 wherein, the cycle time may range from 15 minutes to 60 minutes.

25. The method of claim 24 wherein the treated air through heat, fragrance or atomized air is controlled in sequence and time.
